(19) United States
(12) Patent Application Publication
(10) Pub. No.: US 2026/0139063 A1
(43) Pub. Date: May 21, 2026

(54) PRE-MEDICATION AND IMPROVED TREATMENT REGIMEN

(71) Applicant: MonTa Biosciences ApS, Kgs. Lyngby (DK)

(72) Inventors: Simon Skjøde Jensen, Kgs. Lyngby (DK); Kristoffer Staal Rohrberg, Kgs. Lyngby (DK); Morten Just Petersen, Kgs. Lyngby (DK); Svetlana Panina, Kgs. Lyngby (DK)

(21) Appl. No.: 19/121,856

(22) PCT Filed: Oct. 19, 2023
(86) PCT No.: PCT/EP2023/079090

§ 371 Date: Apr. 17, 2025

(30) Foreign Application Priority Data
Oct. 20, 2022 (EP) 22202704.7

(51) Int. Cl. C07K 16/28; A61K 9/00; A61K 9/1271; A61K 9/51; A61K 31/4745; A61K 31/55; A61K 31/675; A61K 31/7105; A61K 38/04; A61K 39/00; A61P 37/06; B82Y 5/00; C07K 16/24; C12N 15/113

(52) U.S. Cl. CPC C07K 16/2866; A61K 9/0019; A61K 9/1271; A61K 9/5123; A61K 31/4745; A61K 31/55; A61K 31/675; A61K 31/7105; A61K 38/04; A61P 37/06; C07K 16/248; C12N 15/113; A61K 2039/505; B82Y 5/00; C07K 2317/24; C12N 2310/3513

(57) ABSTRACT

The present invention relates to the use of an anti-interleukin-6 agent prior to, simultaneously with, or after a CRS inducing immunotherapeutic agent.

### TECHNICAL FIELD

The present invention relates to the use of an anti-interleukin-6 agent prior to, simultaneously with, or after a CRS inducing immunotherapeutic agent. In particular, the present invention relates to use of tocilizumab prior to, simultaneously with, or after treatment with a TLR7 agonist, such as MBS8(1V270).

### BACKGROUND

Toll-like receptors (TLRs) are a class of receptors expressed on various cell types and play a key role in the innate immune system. Upon activation, TLRs activate signal transduction pathways involved in immune activation. Several mammalian TLRs and a number of their agonists have been identified. For example, guanine and uridine rich single-stranded RNA has been identified as a natural ligand for TLR7. In addition, several low molecular weight activators of TLR7 have been identified, including imidazoquinolines, and purine-like molecules. While TLR stimulation initiates a common signaling cascade (involving the adaptor protein MyD88, the transcription factor NFκB, and proinflammatory and effector cytokines), different TLRs are expressed by different cell types, however, TLR7 is mainly expressed in monocytes, plasmacytoid dendritic cells, myeloid dendritic cells and B-cells and are localized to the endosome membrane.

TLR7 has been shown to play a significant role in the pathogenesis of cancer as well as in the regulation of antiviral immunity. A TLR7 agonist, Aldara (Imiquimod), an imidazoquinoline, has been approved for topical treatment of superficial basal cell carcinoma. Due to their ability to induce robust production of anti-cancer cytokines such as interleukin-12, TLR7 agonists have also been investigated for cancer immunotherapy.

Cytokine release syndrome (CRS) is a form of systemic inflammatory response syndrome (SIRS) that can be triggered by a variety of factors such as infections and certain drugs, for example by certain CRS inducing immunotherapeutic agents, such as TLR agonists, as well as cellular therapies such as CAR-T cell therapies

Tocilizumab, sold under the brand name Actemra among others, is an immunosuppressive drug, used for the treatment of rheumatoid arthritis and systemic juvenile idiopathic arthritis, a severe form of arthritis in children. It is a humanized monoclonal antibody against the interleukin-6 receptor (IL-6R). Interleukin 6 (IL-6) is a cytokine that plays an important role in immune responses and is implicated in the pathogenesis of many diseases. In 2017, the FDA approved tocilizumab for treatment of CRS, commonly observed as a side effect of CAR-T cell therapies.

Treatment of severe diseases, such as cancers, hinges on efficiently managing the desired clinical benefit over any side effects associated with the treatment. Hence, the art is in need of treatment regimens increasing the clinical benefit while minimizing or preventing altogether the side effects.

### SUMMARY

The present inventors have discovered that a combination treatment of a disease, such as cancer, with an anti-interleukin-6 (IL-6) agent and an immunotherapeutic agent which is also a CRS inducing immunotherapeutic agent is surprisingly effective in treating the disease while avoiding or decreasing risk of developing drug-induced CRS when the anti-interleukin-6 (IL-6) agent is administered prior to, simultaneously with, or after the CRS inducing immunotherapeutic agent in a patient who does not present with symptoms of CRS at the time the combination drug is administered. As both the beneficial pharmacological effects and the potential side effects of the CRS inducing immunotherapeutic agent are caused by modulation of the immune system by the CRS inducing immunotherapeutic agent, blocking parts of the immune system to avoid or decrease risk of side effects would likely also decrease the beneficial pharmacological effects. Hence, the surprising beneficial effects of the combination treatment disclosed herein could not have been predicted a priori. Further, decreasing side effects of the CRS inducing immunotherapeutic agent allows for administration of higher doses of said agent, ultimately providing a better therapeutic outcome for the patient.

Hence in one aspect, a combination drug is provided comprising, separately or together, an anti-interleukin-6 (IL-6) agent and a cytokine release syndrome (CRS) inducing immunotherapeutic agent for use in the treatment of a disease in a patient, wherein the anti-IL-6 agent is administered to the patient prior to, simultaneously with, or after the CRS inducing immunotherapeutic agent, wherein the patient does not present with symptoms of CRS.

In one aspect, a combination drug is provided comprising, separately or together, an anti-interleukin-6 (IL-6) agent and a cytokine release syndrome (CRS) inducing immunotherapeutic agent for use in the treatment of a disease in a patient who does not present with symptoms of CRS.

In a further aspect, a method for treatment of a disease in a patient is provided comprising administering an anti-interleukin-6 (IL-6) agent and a CRS inducing immunotherapeutic agent to the patient, wherein the anti-IL-6 agent is administered to the patient prior to, simultaneously with, or after the CRS inducing immunotherapeutic agent, wherein the patient does not present with symptoms of CRS.

In a further aspect, use of an anti-interleukin-6 (IL-6) agent is provided for prevention or reducing risk of developing Cytokine Release Syndrome (CRS) in a patient about to receive, receiving, or who has received a CRS inducing immunotherapeutic agent, wherein the patient does not present with symptoms of CRS.

In a further aspect, use of an anti-interleukin-6 (IL-6) agent is provided for prevention or reducing risk of developing Cytokine Release Syndrome (CRS) in a patient at risk of developing CRS in response to treatment with a CRS inducing immunotherapeutic agent, wherein the anti-interleukin-6 (IL-6) agent is administered prior to, simultaneously with, or after the CRS inducing immunotherapeutic agent, wherein the patient does not present with symptoms of CRS.

In a further aspect, a method for identifying a patient having elevated risk of CRS and subsequently treating the patient is provided, wherein the method comprises;
a. Assessment of one or more biomarkers,
b. Treating the patient with the combination drug as defined herein.

In a further aspect, an anti-interleukin-6 (IL-6) agent for use in the treatment of a disease in a patient is provided, wherein the anti-IL-6 agent is administered to the patient prior to, simultaneously with, or after a CRS inducing immunotherapeutic agent, wherein the patient does not present with symptoms of CRS.

In a further aspect, a kit of parts is provided comprising, separately or together, an anti-IL-6 agent and a CRS inducing immunotherapeutic agent

### DESCRIPTION OF DRAWINGS AND FIGURES

The figures included herein are illustrative and simplified for clarity, and they merely show details which are essential to the understanding of the invention, while other details may have been left out. When using reference numerals in drawings, throughout the specification, claims and drawings the same reference numerals are used for identical or corresponding parts. The figures and drawing include:

FIG. 1: Detection of cytokines in plasma of subjects treated with 0.1 mg/kg MBS8(1V270) without (left panels) or with (right panels) pre-medication with tocilizumab (8 mg/kg). The measurements were done using GLP/GCP compliant multiplex panels purchased from Meso Scale Discovery (MSD) (Cytokine Panel 1 Human kit, Cytokine Panel 2 Human kit and Chemokine Panel 1 Human kit). Data treatment was performed using GraphPad Prism 9 software. Selected biomarkers were IL-6 (FIG. 1A), TNF-α (FIG. 1B), IL-1RA (FIG. 1C), IFN-γ (FIG. 1D), IP-10 (FIG. 1E), IL-1β (FIG. 1F), IL-8 (FIG. 1G) and IL-10 (FIG. 1H).

FIG. 2: Table overview (FIG. 2A) of CRS occurrences in patients were assessed according to CTCAE grading, in patients treated with MBS8(1V270) on 4 occasions on day 1, 8, 15 and 22. Patient (pt) 1-3 were not pretreated with tocilizumab, whereas pt 4-7 were pretreated with tocilizumab prior to dosing with MBS8(1V270). All subjects received MBS8(1V270) at 0.1 mg/kg at each dosing occasion, and the clinical picture observed and rated according to the CTCAE grading shown in FIG. 2B. N/A=Not applicable. Table overview (FIG. 2B) of official CTCAE grading of patients with CRS symptoms as used for grading of CRS severeness for patients in FIG. 2A.CRS grading was done according to CTCAE Version 5.0.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION

The features and advantages of the present invention is readily apparent to a person skilled in the art by the below detailed description of embodiments and examples of the invention with reference to the figures and drawings included herein.

### Definitions

The term “combination drug” in the context of the present disclosure covers a plurality of drugs which can be in separate packings but are administered to the same patient such that the effects exerted by each individual drug collectively provide an improved treatment for the patient over monotherapy with any of the individual drugs.

The term “anti-interleukin-6 agent” in the context of the present disclosure covers an agent that inhibits the functional output of IL-6, either directly or indirectly. Exemplary classes of anti-interleukin-6 agents can be an interleukin-6 receptor (IL-6R) blocking agent, an interleukin-6 (IL-6) blocking agent, or an interleukin-6 (IL-6) signaling blocking agent.

The term “liquid tumor” in the context of the present disclosure covers a malignant tumor that originates from myeloid or lymphoid cells, such as leukemias and lymphomas.

The term “CRS inducing immunotherapeutic agent” in the context of the present disclosure covers an immunotherapeutic agent which at therapeutically relevant doses can lead to CRS. For clinical candidates undergoing clinical development, for example for anti-cancer immunotherapeutic agents, incidence of CRS is documented, and whether or not an immunotherapeutic agent is classified as a CRS inducing immunotherapeutic agent is known at the latest subsequent to clinical studies. Often, the CRS inducing immunotherapeutic agent of the present disclosure is selected from the group consisting of: a toll-like receptor (TLR) agonist, chimeric antigen receptor (CAR) T-cell therapy, and a bispecific antibody, such as blinatumomab.

The term “DSPE-PEG2000” in the context of the present disclosure covers 1,2-distearoyl-sn-glycero-3-phosphoethanolamine-N-[methoxy(polyethylene glycol)-2000], for example 1,2-distearoyl-sn-glycero-3-phosphoethanolamine-N-[methoxy(polyethylene glycol)-2000](sodium salt), which is also referred to as [N-(Carbonyl-methoxypolyethylene glycol-2000)-1,2-distearoyl-sn-glycero-3-phosphoethanolamine, sodium salt]

The term “administration” in the context of the present disclosure refers to the act of providing a medicament referred to herein, such as an anti-IL-6 agent and/or a CRS inducing immunotherapeutic agent, to a patient. In the context of administration at a certain point in time, i.e. within a predefined time interval, the time interval refers to the start of administration. In the context of administration over a period of time, such as by infusion, the time of administration refers to the time point when infusion is started unless otherwise specified herein.

### Combination Drug

The present disclosure provides a combination drug comprising, separately or together, an anti-interleukin-6 (IL-6) agent and a cytokine release syndrome (CRS) inducing immunotherapeutic agent for use in the treatment of a disease in a patient, wherein the anti-IL-6 agent is administered to the patient prior to, simultaneously with, or after the CRS inducing immunotherapeutic agent. In particular, for patients not presenting with symptoms of CRS. In a preferred embodiment, the disease is a cancer, and the combination drug disclosed herein can be used in an improved method for treatment of cancer. By decreasing side effects of the CRS inducing immunotherapeutic agent, higher doses of said agent can be administered, ultimately providing a better therapeutic outcome for the patient. As demonstrated in Example 2 of the present application, no CRS was observed at any dosing occasion in the patients which were pretreated with the anti-IL-6 agent, tocilizumab, whereas the patients that were not pretreated with tocilizumab all experienced CRS during the 4 administrations. In total CRS was experienced at 0 of the 14 dosing occasions (0%) for the tocilizumab premedicated patients, but in 7 out of 12 occasions (58%) in the non-premedicated patients. The results demonstrated in Example 2 support the significant benefit obtained by the combination drug of the present disclosure.

### Patients

The present disclosure further provides a method involving a patient at risk of developing cytokine release syndrome (CRS), such as drug induced CRS. The incidence of CRS in patients receiving cancer immunotherapy varies widely depending on the type of immunotherapeutic agent. The onset of CRS can occur within a few days, and in the case of CAR T cell therapy, up to several weeks after infusion of the drug. With most conventional monoclonal antibodies, the incidence of CRS is relatively low, whereas T cell-engaging cancer immunotherapies carry a particularly high risk of CRS.

Interleukin 6 (IL-6) appears to hold a key role in CRS pathophysiology since highly elevated IL-6 levels are seen in patients with CRS. IL-6 contributes to many of the key symptoms of CRS. Via trans-signaling IL-6 leads to characteristic symptoms of severe CRS, i.e. vascular leakage, and activation of the complement and coagulation cascade inducing disseminated intravascular coagulation (DIC). In addition, IL-6 likely contributes to cardiomyopathy that is often observed in patients with CRS by promoting myocardial dysfunction.

In some embodiments, the patient to be treated by the combination drug suffers from a condition that increases the risk of developing CRS. In some embodiments, the condition is selected from the group consisting of: a cancer, and an autoimmune disease.

The patients treated by the combination drug of the present disclosure have not yet presented with symptoms of CRS. In this context, a patient is treated who does not present with symptoms of CRS at the time the combination drug of the present disclosure is administered to the patient. In the present context, “patients presenting with symptoms of CRS” and “patients with CRS” are used interchangeably.

### Anti-Interleukin-6 Agents

In some embodiments, the present disclosure provides an anti-interleukin-6 agent which is an interleukin-6 receptor (IL-6R) blocking agent, an interleukin-6 (IL-6) blocking agent, or an interleukin-6 (IL-6) signaling blocking agent.

In some embodiments, the anti-interleukin-6 agent is a monoclonal antibody against the interleukin-6 receptor (IL-6R), such as a humanized monoclonal antibody against IL-6R.

In some embodiments, the anti-interleukin-6 agent is a monoclonal antibody against interleukin-6, such as a humanized monoclonal antibody against IL-6.

In some embodiments, the combination drug of the disclosure is provided where the anti-IL-6 agent is selected from the group consisting of: tocilizumab (CAS: 375823-41-9), siltuximab (CAS: 541502-14-1), sarilumab (CAS: 1189541-98-7), olokizumab (CAS: 1007223-17-7), elsilimomab (CAS: 468715-71-1), clazakizumab (CAS: 1236278-28-6), and sirukumab (CAS: 1194585-53-9).

In some embodiments, the CRS inducing immunotherapeutic agent is selected from the group consisting of: a toll-like receptor (TLR) agonist, chimeric antigen receptor (CAR) T-cell therapy, and a bispecific antibody, such as blinatumomab.

### Toll-Like Receptor (TLR) Agonists

In some embodiments, the CRS inducing immunotherapeutic agent is a toll-like receptor (TLR) agonist.

In some embodiments, the CRS inducing immunotherapeutic agent is a toll-like receptor (TLR) agonist selected from the group consisting of: a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, a TLR10 agonist, a TLR11 agonist, and a TLR12 agonist.

In some embodiments, the CRS inducing immunotherapeutic agent is selected from the group consisting of: a TLR3 agonist, a TLR7 agonist, a TLR8 agonist, and a TLR9 agonist, or a dual agonist thereof, such as a dual TLR7/8 agonist.

In some embodiments, the TLR7 agonist is selected from the group consisting of: 852A; BNT411; DSP-0509; LHC165; NJH395; Resiquimod; RO7119929; TQ-A3334; and BDC-1001.

In some embodiments, the TLR7 agonist is selected from the group consisting of:

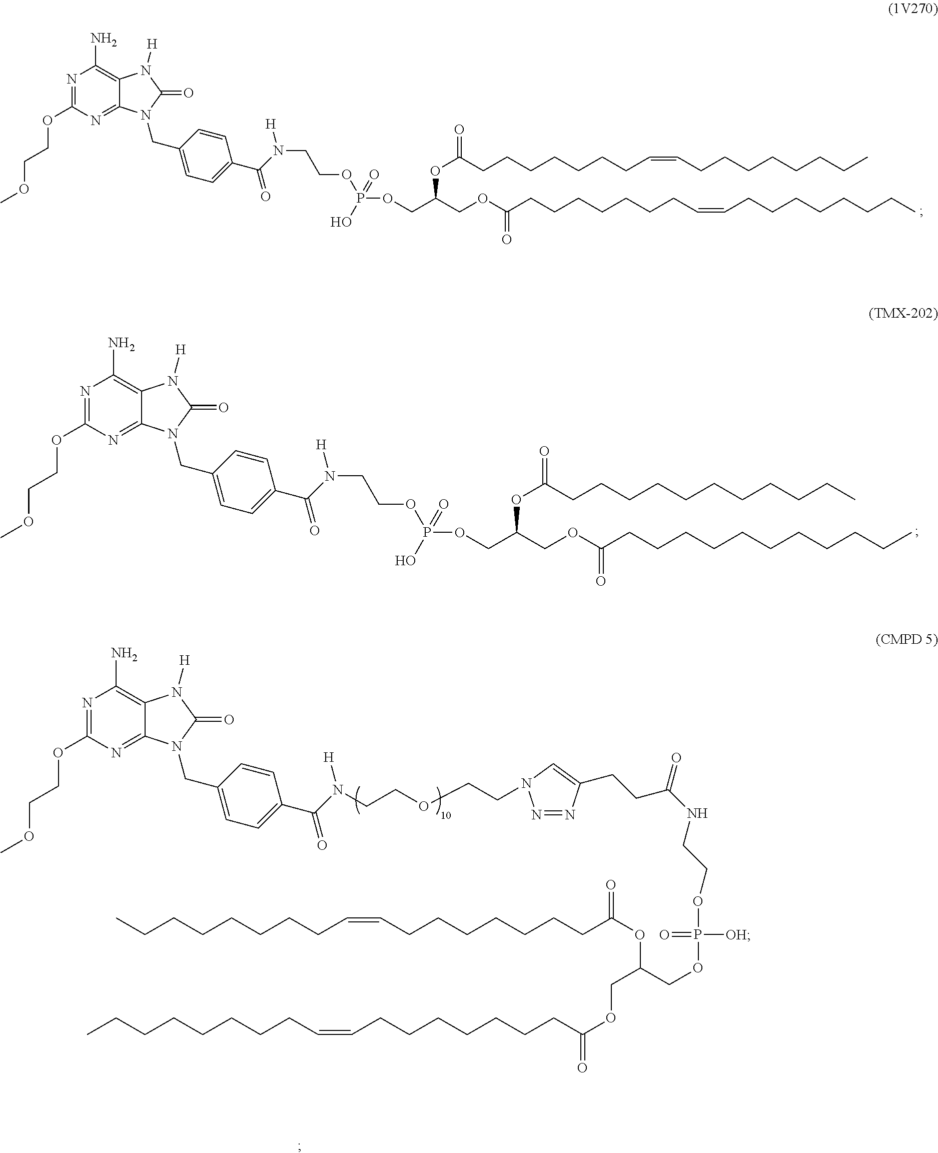

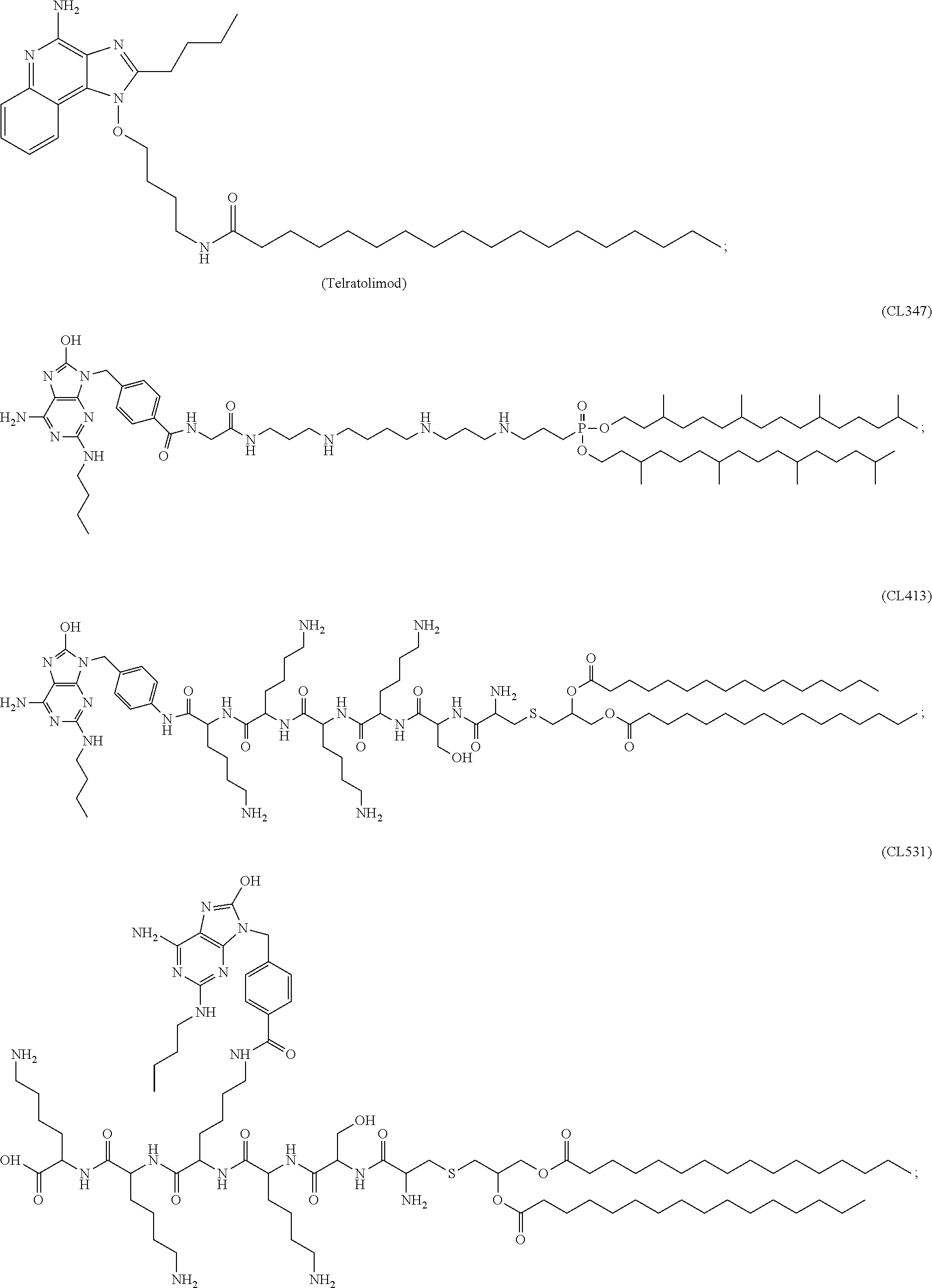

a tautomer thereof, and a pharmaceutically acceptable salt thereof.

In some embodiments, the combination drug is provided, wherein the TLR8 agonist is selected from the group consisting of: CV8102; NKTR-262; and Motolimod.

In other embodiments, the CRS-inducing immunotherapeutic agent is a dual agonist of certain TLR's. In particular the dual agonist is a TLR7/8 agonist, i.e. dual agonist of TLR7 and 8, for example the long-acting prodrug of resiquimod, TransCon TLR7/8 Agonist or 3M-052.

### Administration

In some embodiments, the CRS inducing immunotherapeutic agent is formulated for parenteral or oral administration.

In some embodiments, the CRS inducing immunotherapeutic agent is formulated for intravenous administration, subcutaneous administration, intraperitoneal administration, and/or intratumoral administration.

### Formulation

In some embodiments, the CRS inducing immunotherapeutic agent is formulated as a micelle or a liposome. In some embodiments, the CRS inducing immunotherapeutic agent is formulated as a micelle having a diameter of from 5 nm to 39 nm.

In some embodiments, CRS inducing immunotherapeutic agent is formulated as a micelle which is MBS8, comprising
i) 1,2-distearyl-sn-glycero-3-phosphoethanolamine-N-[methoxy(polyethylene glycol)-2000](DSPE-PEG2000); and
ii) the TLR7 agonist of formula (IA),

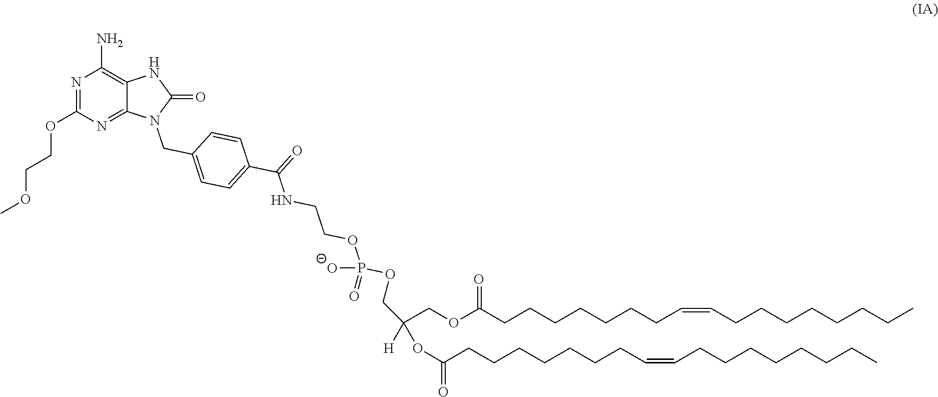 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]

or
a pharmaceutically acceptable salt or solvate thereof;
wherein the molar ratio of DSPE-PEG2000 to the TLR7 agonist 90:10. The TLR7 agonist of formula (IA) also includes tautomers thereof.

### Diseases

In some embodiments, the disease treated by the method, or the combination drug disclosed herein is a cancer.

In some embodiments, the cancer is selected from the group consisting of: a solid tumor and a liquid tumor.

In some embodiments, the cancer is selected from the group consisting of: lung cancer, breast cancer, prostate cancer, head and neck cancer, leukemia, ovarian, lymphoma and melanoma.

### Timing of Administration

In some embodiments, the anti-IL-6 agent is administered within a predefined time interval from prior administration of the CRS inducing immunotherapeutic agent to after administration of the CRS inducing immunotherapeutic agent in a patient which does not present with symptoms of CRS. In some embodiments, the combination drug is administered to the patient prior to development of symptoms of CRS. Administering the anti-IL-6 agent within a predefined time interval prior to the patient presents with CRS symptoms successfully decreases the incidence of CRS as demonstrated in Example 2.

In some embodiments, the anti-IL-6 agent is administered within a predefined time interval prior to, simultaneously with, or after the CRS inducing immunotherapeutic agent. In some embodiments, the anti-IL-6 agent is administered within a predefined time interval prior to the CRS inducing immunotherapeutic agent.

In some embodiments, the anti-IL-6 agent is administered shortly after the CRS inducing immunotherapeutic agent but prior to appearance of CRS.

In some embodiments, the predefined time interval is from 240 minutes prior to administration of the CRS inducing immunotherapeutic agent to 240 minutes after administration of the CRS inducing immunotherapeutic agent.

In some embodiments, the predefined time interval is from 240 minutes to 10 minutes prior to administration of the CRS inducing immunotherapeutic agent, such as from 220 minutes to 10 minutes, such as from 200 minutes to 20 minutes, such as from 180 minutes to 30 minutes, such as from 160 minutes to 40 minutes, such as from 140 to 50 minutes, such as from 120 minutes to 60 minutes prior to administration of the CRS inducing immunotherapeutic agent.

In some embodiments, the anti-IL-6 agent is administered to the patient no later than 240 minutes after the CRS inducing immunotherapeutic agent, such as no later than 220 minutes, such as no later than 200 minutes, such as no later than 180 minutes, such as no later than 160 minutes, such as no later than 140 minutes, such as no later than 120 minutes, such as no later than 100 minutes, such as no later than 80 minutes, such as no later than 60 minutes, such as no later than 40 minutes, such as no later than 20 minutes after the CRS inducing immunotherapeutic agent.

In some embodiments, the CRS inducing immunotherapeutic agent is administered to the patient over a time course of approximately 2 hours, and the anti-IL-6 agent is administered simultaneously with the CRS inducing immunotherapeutic agent or administered within approximately 1 hour after said time course, such as approximately immediately after. In this context, the term “administration” refers to the act of providing the anti-IL-6 agent and/or a CRS inducing immunotherapeutic agent, to a patient. In this context of administration at a certain point in time, i.e. within a predefined time interval, the time interval refers to the start of administration. In the context of administration over a period of time, such as by infusion, the administration refers to the time point when infusion is started.

In some embodiments, the anti-interleukin-6 (IL-6) agent is tocilizumab (CAS: 375823-41-9), and the CRS inducing immunotherapeutic agent is MBS8 and wherein tocilizumab is administered to the patient from 240 minutes prior to administration of MBS8 to 180 minutes after administration of MBS8.

In some embodiments, the anti-interleukin-6 (IL-6) agent is tocilizumab (CAS: 375823-41-9), and the CRS inducing immunotherapeutic agent is MBS8 and wherein tocilizumab is administered to the patient from 240 minutes prior to administration of MBS8 to 180 minutes after administration of MBS8, wherein MBS8 is administered to the patient in a dosage of at least 0.1 mg/kg.

In some embodiments, the anti-interleukin-6 (IL-6) agent is tocilizumab (CAS: 375823-41-9), and the CRS inducing immunotherapeutic agent is MBS8 and wherein MBS8 is administered by infusion to the patient over a time course of approximately 2 hours and tocilizumab is administered within approximately 1 hour after said time course, such as approximately immediately after.

In preferred embodiments, the anti-interleukin-6 (IL-6) agent is tocilizumab (CAS: 375823-41-9), and the CRS inducing immunotherapeutic agent is MBS8 as defined herein, wherein tocilizumab is administered to the patient from 240 minutes to 10 minutes prior to administration of MBS8. In some embodiments, the disease in this context is cancer and the treatment effectively reduces incidence of CRS while effectively treating the cancer.

In preferred embodiments, the anti-interleukin-6 (IL-6) agent is tocilizumab (CAS: 375823-41-9), and the CRS inducing immunotherapeutic agent is MBS8, wherein tocilizumab is administered to the patient from 240 minutes to 10 minutes prior to administration of MBS8, wherein MBS8 is administered to the patient in a dosage of at least 0.1 mg/kg. In some embodiments pertaining to this aspect, the disease is cancer and the treatment effectively reduces incidence of CRS while effectively treating the cancer.

### Methods and Uses

In the context of the present disclosure, all aspects provided for the combination drug for use disclosed herein are also included for the related methods for use. As such, the present disclosure provides a method for treatment of a disease in a patient comprising administering an anti-interleukin-6 (IL-6) agent and a CRS inducing immunotherapeutic agent to the patient, wherein the anti-interleukin-6 (IL-6) agent is administered to the patient prior to or simultaneous with the CRS inducing immunotherapeutic agent.

In some embodiments, use of an anti-interleukin-6 (IL-6) agent is provided for prevention or reducing risk of developing Cytokine Release Syndrome (CRS) in a patient about to receive or receiving a CRS inducing immunotherapeutic agent. This effect of the disclosed use is credibly demonstrated in at least Example 2 of the present application. The timing of administration in these cases are such that the anti-IL-6 agent is administered within a predefined time interval prior to administration of a CRS inducing immunotherapeutic agent in a patient. The relevant patients for said use are in some embodiments patients that expected to be prone to drug induced CRS upon receiving a CRS inducing immunotherapeutic agent.

In some embodiments, use of an anti-interleukin-6 (IL-6) agent is provided for prevention or reducing risk of developing Cytokine Release Syndrome (CRS) in a patient at risk of developing CRS in response to treatment with a CRS inducing immunotherapeutic agent, wherein the anti-interleukin-6 (IL-6) agent is administered prior to the CRS inducing immunotherapeutic agent.

In some embodiments, the present disclosure provides a method for identifying a patient having elevated risk of CRS and subsequently treating the patient, wherein the method comprises;
a. Assessment of one or more biomarkers,
b. Treating the patient with the combination drug.

In some embodiments, an anti-interleukin-6 (IL-6) agent is provided for use in the treatment of a disease in a patient who does not present with symptoms of CRS, wherein the anti-IL-6 agent is administered to the patient prior to, simultaneously with, or after a CRS inducing immunotherapeutic agent, such as MBS8.

### Kit of Parts

In some embodiments, a kit of parts is provided comprising, separately or together, an anti-IL-6 agent and a CRS inducing immunotherapeutic agent.

### Combination Therapy

As the present disclosure in preferred embodiments relates to treatment of cancers, administration of the combination drug disclosed herein effectively combines with one or more further anti-cancer agents. Hence in some aspects, the present disclosure provides a combination drug for use in combination with one or more further anti-cancer agents and/or radiotherapy and/or a further cancer treatment modality.

In some embodiments, the treatment of cancer is enhanced by combination of existing treatments like monoclonal antibodies (Trastuzumab, Rituximab, Cetuximab), radiotherapy, chemotherapy or immune checkpoint inhibitors like Pembrolizumab, Ipilimumab. Hence, in one embodiment, the treatment of cancer is a combination treatment further comprising administering a monoclonal antibody to the patient suffering from cancer.

Certain types of chemotherapy are especially relevant for combination with TLR7 assets; these are chemotherapy compounds that induce what is called “immunogenic cell death” (ICD). In one embodiment, the treatment of cancer involves the combination drug disclosed herein in a combination treatment further comprising administering a chemotherapeutic agent, such as doxorubicin or doxil. In one embodiment, the chemotherapeutic agent is selected from the group consisting of Doxorubicin, Doxil, Epirubicin, Cyclophosphamide, Bortezomib, and Oxaliplatin.

In some embodiments, the treatment of cancer is a combination treatment further comprising administering immune checkpoint inhibitors, such as monoclonal antibodies targeting PD-1 or PD-L1, such as α-PD-1 or α-PD-L1, for example Atezolizumab, Avelumab, Durvalumab, Nivolumab, Tislelizumab or Pembrolizumab. Preferably the immune checkpoint inhibitor is an α-PD-1, such as Nivolumab or Pembrolizumab. In a preferred embodiment, the combination drug is used with a further anti-cancer agent selected from: Nivolumab or Pembrolizumab.

In some embodiments, the combination drug of the present disclosure is administered in combination with a therapeutic antibody targeting the PD-1/PD-L1 pathway to a cancer patient in need of treatment. In one embodiment, the therapeutic antibody targeting the PD-1/PD-L1 pathway is selected from the group consisting of: Atezolizumab, Avelumab, Durvalumab, Nivolumab, Pembrolizumab, Spartalizumab/PDR001, Tislelizumab, BCD-100, TSR-042, Camrelizumab, IB1308, KN035, and CS1001.

In some embodiments, the combination drug of the present disclosure is administered to a cancer patient in combination with a monoclonal antibody selected from the group consisting of: Ublituximab, Obinutuzumab, Ofatumumab, Ibritumomab, Tiuxetan, Rituximab, Tositumomab, Depatuxizumab mafodotin, Necitumumab, Panitumumab, Cetuximab, Trastuzumab, Trastuzumab-dkst, Trastuzumab emtansine, BAT8001, Pertuzumab, Margetuximab, Trastuzumab deruxtecan, Trastuzumab duocarmazine, Daratumumab, and Isatuximab.

In some embodiments, the combination drug is administered in combination with an antibody targeting CD47, such as Magrolimab. In some embodiments, the combination drug is administered with one or more of the anti-cancer agents referred to herein and radiotherapy.

In some embodiments, the combination drug is administered in combination with radiotherapy.

### Items

1. A combination drug comprising, separately or together, an anti-interleukin-6 (IL-6) agent and a CRS inducing immunotherapeutic agent for use in the treatment of a disease in a patient, wherein the anti-IL-6 agent is administered to the patient prior to, simultaneously with, or after the CRS inducing immunotherapeutic agent, wherein the patient does not present with symptoms of CRS.

2. The combination drug for use according to any one of the preceding items, wherein the patient is at risk of developing cytokine release syndrome (CRS), such as drug induced CRS.

3. The combination drug for use according to any one of the preceding items, wherein the patient suffers from a condition that increases the risk of developing CRS.

4. The combination drug for use according to any one of the preceding items, wherein the patient suffers from a condition selected from the group consisting of: a cancer, and an autoimmune disease.

5. The combination drug for use according to any one of the preceding items, wherein the anti-interleukin-6 agent is an interleukin-6 receptor (IL-6R) blocking agent, an interleukin-6 (IL-6) blocking agent, or an interleukin-6 (IL-6) signalling blocking agent.

6. The combination drug for use according to any one of the preceding items, wherein the anti-interleukin-6 agent is a monoclonal antibody against the interleukin-6 receptor (IL-6R), such as a humanized monoclonal antibody against IL-6R.

7. The combination drug for use according to any one of items 1-5, wherein the anti-interleukin-6 agent is a monoclonal antibody against interleukin-6, such as a humanized monoclonal antibody against IL-6.

8. The combination drug for use according to any one of the preceding items, wherein the anti-IL-6 agent is selected from the group consisting of: tocilizumab (CAS: 375823-41-9), siltuximab (CAS: 541502-14-1), sarilumab (CAS: 1189541-98-7), olokizumab (CAS: 1007223-17-7), elsilimomab (CAS: 468715-71-1), clazakizumab (CAS: 1236278-28-6), and sirukumab (CAS: 1194585-53-9).

9. The combination drug for use according to any one of the preceding items, wherein the CRS inducing immunotherapeutic agent is selected from the group consisting of: a toll-like receptor (TLR) agonist, chimeric antigen receptor (CAR) T-cell therapy, and a bispecific antibody, such as blinatumomab.

10. The combination drug for use according to any one of the preceding items, wherein the CRS inducing immunotherapeutic agent is a toll-like receptor (TLR) agonist.

11. The combination drug for use according to any one of the preceding items, wherein the CRS inducing immunotherapeutic agent is a toll-like receptor (TLR) agonist selected from the group consisting of: a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, a TLR10 agonist, a TLR11 agonist, and a TLR12 agonist.

12. The combination drug for use according to any one of the preceding items, wherein the CRS inducing immunotherapeutic agent is selected from the group consisting of: a TLR3 agonist, a TLR7 agonist, a TLR8 agonist, and a TLR9 agonist, or a dual agonist thereof, such as a dual TLR7/8 agonist.

13. The combination drug for use according to item 11, wherein the TLR7 agonist is selected from the group consisting of: 852A; BNT411; DSP-0509; LHC165; NJH395; Resiquimod; RO7119929; TQ-A3334; and BDC-1001.

14. The combination drug for use according to item 11, wherein the TLR7 agonist is selected from the group consisting of:

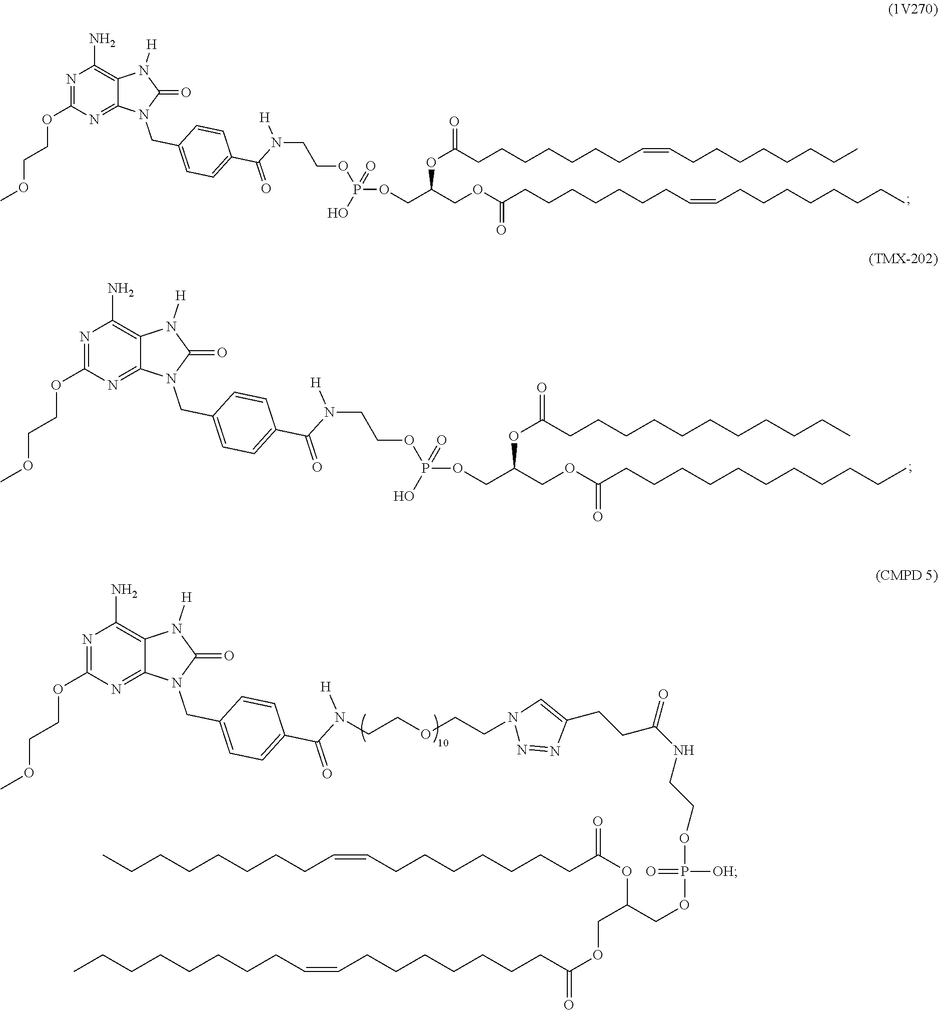

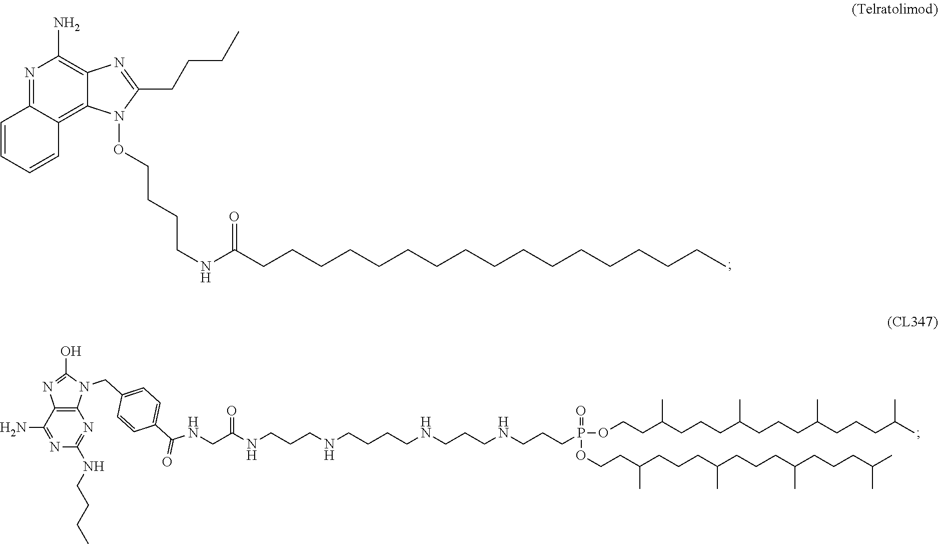 [SMILES: CCCCCCCCCCCCCCCCCC(=O)NCCCCOn1c(CCCC)nc2c(N)nc3ccccc3c21 ; CCCCNc1nc(N)c2nc(O)n(Cc3ccc(C(=O)NCC(=O)NCCCNCCCCNCCCNCCCP(=O)(OCCC(C)CCCC(C)CCCC(C)CCCC(C)C)OCCC(C)CCCC(C)CCCC(C)CCCC(C)C)cc3)c2n1]

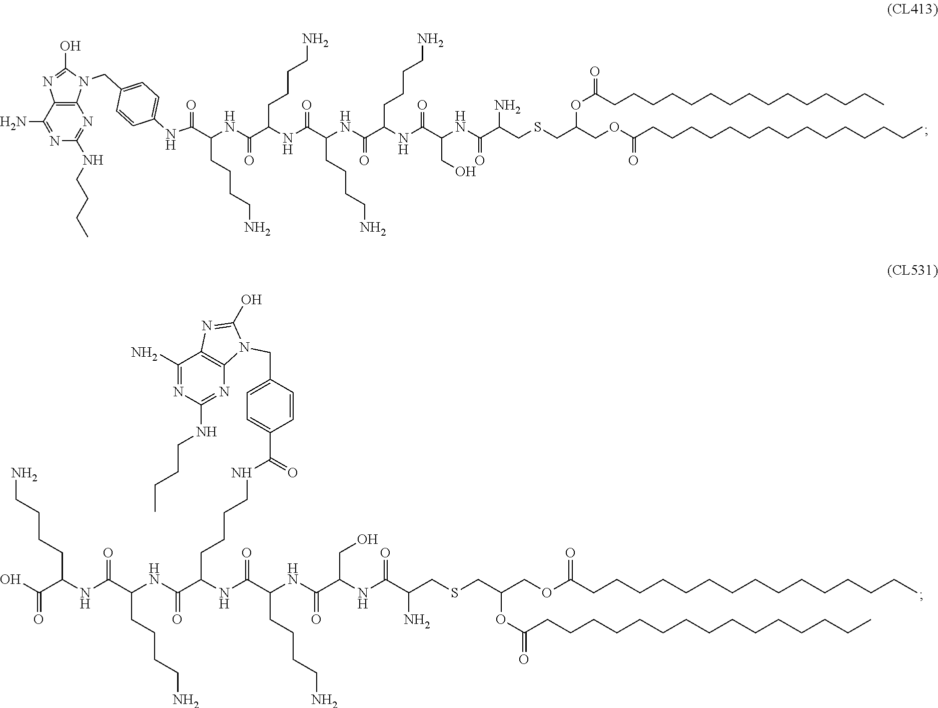 [SMILES: CCCCCCCCCCCCCCCC(=O)OCC(CSCC(N)C(=O)NC(CO)C(=O)NC(CCCCN)C(=O)NC(CCCCN)C(=O)NC(CCCCN)C(=O)NC(CCCCN)C(=O)Nc1ccc(Cn2c(O)nc3c(N)nc(NCCCC)nc32)cc1)OC(=O)CCCCCCCCCCCCCCC ; CCCCCCCCCCCCCCCC(=O)OCC(CSCC(N)C(=O)NC(CO)C(=O)NC(CCCCN)C(=O)NC(CCCCNC(=O)c1ccc(Cn2c(O)nc3c(N)nc(NCCCC)nc32)cc1)C(=O)NC(CCCCN)C(=O)NC(CCCCN)C(=O)O)OC(=O)CCCCCCCCCCCCCCC]

a tautomer thereof, and a pharmaceutically acceptable salt thereof.

15. The combination drug for use according to item 11, wherein the TLR8 agonist is selected from the group consisting of: CV8102; NKTR-262; and Motolimod.

16. The combination drug for use according to 11, wherein the dual TLR7/8 agonist is the long-acting prodrug of resiquimod, TransCon TLR7/8 Agonist or 3M-052.

17. The combination drug for use according to any one of the preceding items, wherein the CRS inducing immunotherapeutic agent is formulated for parenteral or oral administration.

18. The combination drug for use according to any one of the preceding items, wherein the CRS inducing immunotherapeutic agent is formulated for intravenous administration, subcutaneous administration, intraperitoneal administration, and/or intratumoral administration.

19. The combination drug for use according to any one of the preceding items, wherein the CRS inducing immunotherapeutic agent is formulated as a micelle or a liposome, optionally wherein the micelle has a diameter of from 5 nm to 39 nm.

20. The combination drug for use according to item 19, wherein the micelle is MBS8, comprising
i) 1,2-distearyl-sn-glycero-3-phosphoethanolamine-N-[methoxy(polyethylene glycol)-2000](DSPE-PEG2000); and
ii) the TLR7 agonist of formula (IA),

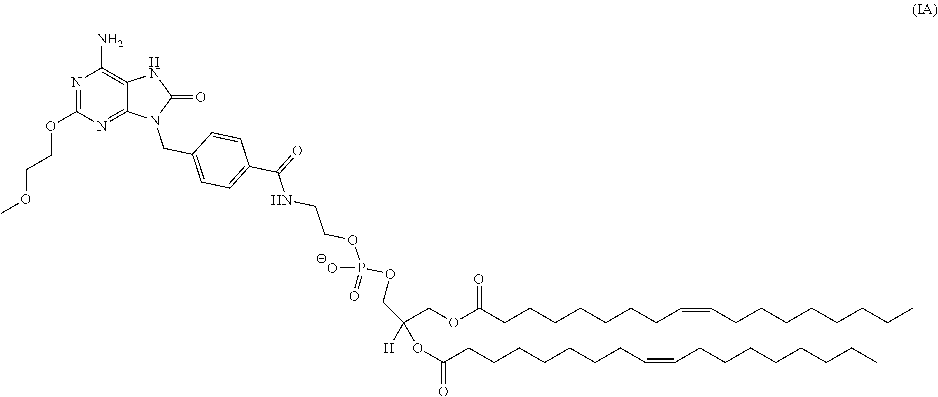 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]

or a pharmaceutically acceptable salt or solvate thereof;
wherein the molar ratio of DSPE-PEG2000 to the TLR7 agonist 90:10.

21. The combination drug for use according to any one of the preceding items, wherein the disease is a cancer.

22. The combination drug for use according to any one of the preceding items, wherein the cancer is selected from the group consisting of: a solid tumor and a liquid tumor.

23. The combination drug for use according to any one of the preceding items, wherein the cancer is selected from the group consisting of: lung cancer, breast cancer, prostate cancer, head and neck cancer, leukemia, ovarian, lymphoma and melanoma.

24. The combination drug for use according to any one of the preceding items, wherein the anti-IL-6 agent is administered simultaneously with the CRS inducing immunotherapeutic agent.

25. The combination drug for use according to any one of the preceding items, wherein the anti-IL-6 agent is administered within a predefined time interval prior to, simultaneously with, or after the CRS inducing immunotherapeutic agent.

26. The combination drug for use according to any one of the preceding items, wherein the anti-IL-6 agent is administered within a predefined time interval prior to the CRS inducing immunotherapeutic agent.

27. The combination drug for use according to item 25, wherein the predefined time interval is from 240 minutes prior to administration of the CRS inducing immunotherapeutic agent to 240 minutes after administration of the CRS inducing immunotherapeutic agent.

28. The combination drug for use according to item 25, wherein the predefined time interval is from 240 minutes to 10 minutes prior to administration of the CRS inducing immunotherapeutic agent, such as from 220 minutes to 10 minutes, such as from 200 minutes to 20 minutes, such as from 180 minutes to 30 minutes, such as from 160 minutes to 40 minutes, such as from 140 to 50 minutes, such as from 120 minutes to 60 minutes prior to administration of the CRS inducing immunotherapeutic agent.

29. The combination drug for use according to item 25, wherein the anti-IL-6 agent is administered to the patient no later than 240 minutes after the CRS inducing immunotherapeutic agent, such as no later than 220 minutes, such as no later than 200 minutes, such as no later than 180 minutes, such as no later than 160 minutes, such as no later than 140 minutes, such as no later than 120 minutes, such as no later than 100 minutes, such as no later than 80 minutes, such as no later than 60 minutes, such as no later than 40 minutes, such as no later than 20 minutes after the CRS inducing immunotherapeutic agent.

30. The combination drug for use according to item 25, wherein the CRS inducing immunotherapeutic agent is administered to the patient over a time course of approximately 2 hours, and the anti-IL-6 agent is administered simultaneously with the CRS inducing immunotherapeutic agent or administered within approximately 1 hour after said time course, such as approximately immediately after.

31. The combination drug for use according to any one of the preceding items, wherein the CRS inducing immunotherapeutic agent is administered to the patient in a dosage of at least 0.1 mg/kg.

32. The combination drug for use according to any one of the preceding items, wherein the anti-interleukin-6 (IL-6) agent is tocilizumab (CAS: 375823-41-9), and the CRS inducing immunotherapeutic agent is MBS8 and wherein tocilizumab is administered to the patient from 240 minutes prior to administration of MBS8 to 180 minutes after administration of MBS8.

33. The combination drug for use according to any one of the preceding items, wherein the anti-interleukin-6 (IL-6) agent is tocilizumab (CAS: 375823-41-9), and the CRS inducing immunotherapeutic agent is MBS8 and wherein tocilizumab is administered to the patient from 240 minutes prior to administration of MBS8 to 180 minutes after administration of MBS8, wherein MBS8 is administered to the patient in a dosage of at least 0.1 mg/kg.

34. The combination drug for use according to any one of the preceding items, wherein the anti-interleukin-6 (IL-6) agent is tocilizumab (CAS: 375823-41-9), and the CRS inducing immunotherapeutic agent is MBS8 and wherein MBS8 is administered by infusion to the patient over a time course of approximately 2 hours and tocilizumab is administered within approximately 1 hour after said time course, such as approximately immediately after.

35. A method for treatment of a disease in a patient comprising administering an anti-interleukin-6 (IL-6) agent and an CRS inducing immunotherapeutic agent to the patient, wherein the anti-IL-6 agent is administered to the patient prior to, simultaneously with, or after the CRS inducing immunotherapeutic agent, wherein the patient does not present with symptoms of CRS.

36. Use of an anti-interleukin-6 (IL-6) agent for prevention or reducing risk of developing Cytokine Release Syndrome (CRS) in a patient about to receive, receiving, or who has received a CRS inducing immunotherapeutic agent, wherein the patient does not present with symptoms of CRS.

37. Use of an anti-interleukin-6 (IL-6) agent for prevention or reducing risk of developing Cytokine Release Syndrome (CRS) in a patient at risk of developing CRS in response to treatment with an CRS inducing immunotherapeutic agent, wherein the anti-interleukin-6 (IL-6) agent is administered prior to, simultaneously with, or after the CRS inducing immunotherapeutic agent, wherein the patient does not present with symptoms of CRS.

38. A method for identifying a patient having elevated risk of CRS and subsequently treating the patient, wherein the method comprises;
a. Assessment of one or more biomarkers,
b. Treating the patient with the combination drug as defined in item 1.

39. An anti-interleukin-6 (IL-6) agent for use in the treatment of a disease in a patient who does not present with symptoms of CRS, wherein the anti-IL-6 agent is administered to the patient prior to, simultaneously with, or after an CRS inducing immunotherapeutic agent.

40. A kit of parts comprising, separately or together, an anti-IL-6 agent and an CRS inducing immunotherapeutic agent.

### EXAMPLES

### Example 1: Clinical Biomarkers

MBS8(1V270) was administered to all subjects at 0.1 mg/kg by IV infusion for a duration of 2 hours for four dosing occasions. Infusions were given one week apart on Days 1, 8, 15, and 22. Patients pretreated with tocilizumab were administered tocilizumab (8 mg/kg) prior to MBS8(1V270) infusion on the first dosing occasion (day 1) as a CRS preventive measure. The biomarkers were measured using GLP/GCP compliant and validated multiplex panels purchased from MSD in a quantitative procedure to measure these cytokines and chemokines.

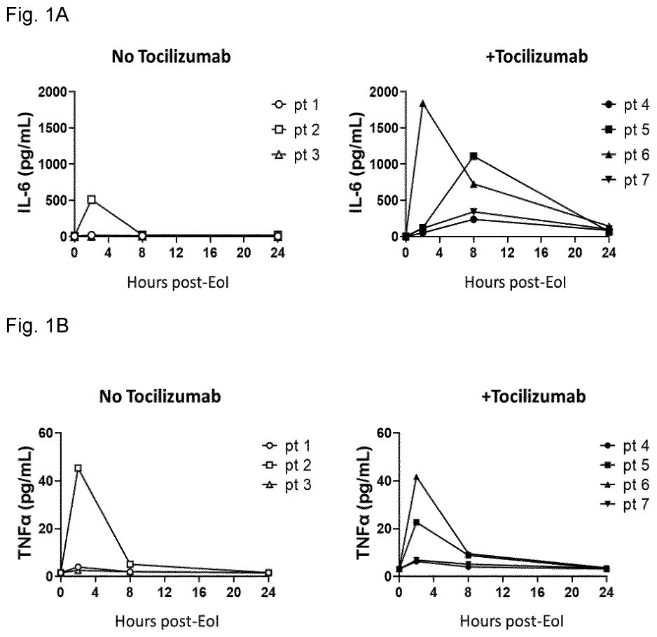

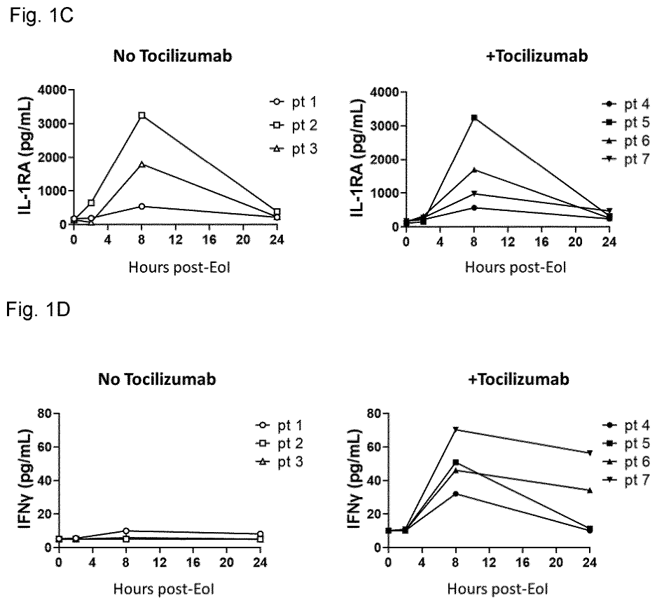

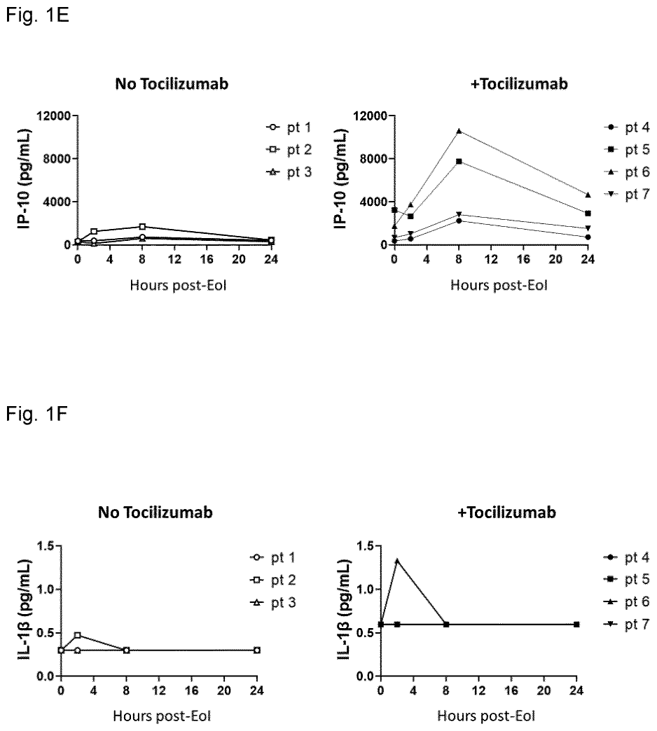

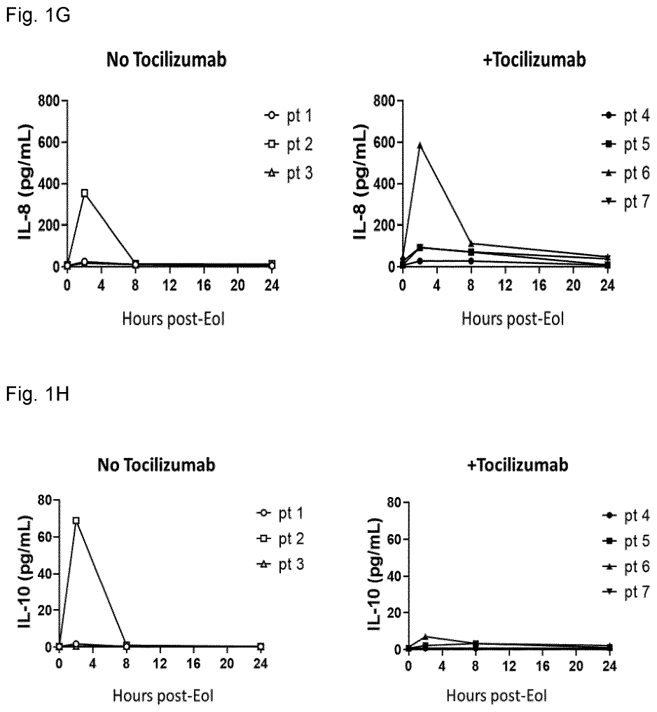

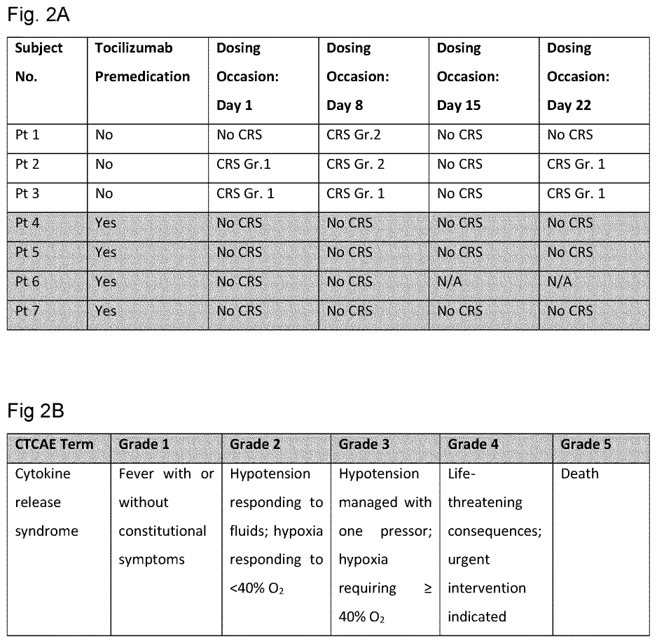

The following plasma biomarkers were identified in this clinical study: safety-related (IL-1β, IL-8, IL-6, TNF-α and IL-10) and pharmacodynamics biomarkers (IFN-γ, MCP-1, IP-10, IL-1RA and IL-12p70) (FIG. 1A-1H). Blood samples were taken at pre-dose (0), 2 h, 8 h, and 24 h post-end of infusion and processed to plasma and frozen for later biomarker analyses.

Conclusion: The present example shows that the use of tocilizumab pre-medication does not downregulate cytokine and chemokine levels expected to play a role for the anti-tumor activity of MBS8(1V270). Some biomarkers show even higher levels after tocilizumab premedication than without, e.g. IL-6, IFN-γ and IP-10, These data show that tocilizumab does not interfere with the MoA of MBS8(1V270) and can be safely used in patients for prevention of CRS without compromising the MoA of MBS8(1V270).

### Example 2: CRS Prevention by Tocilizumab Pre-Medication

MBS8(1V270) was administered to all subjects at 0.1 mg/kg by IV infusion for a duration of 2 hours for four dosing occasions. Infusions were given one week apart on Days 1, 8, 15, and 22. Patients pretreated with tocilizumab were administered tocilizumab (8 mg/kg) prior to MBS8(1V270) infusion on the first dosing occasion as a CRS preventive measure. No clinical signs of CRS were observed in any of the tocilizumab pretreated subjects (pt 4-7), supporting use of tocilizumab as a preventive measure to avoid CRS (Table 1). Seven subjects presenting with advanced solid tumors were treated with MBS8(1V270) at 0.1 mg/kg once weekly on four dosing occasions as described in Example 1. Patient 1-3 did not receive tocilizumab and patient 4-7 received tocilizumab as premedication at the first dosing occasion (Day 1). Tocilizumab was administered as a one-hour intravenous infusion at 8 mg/kg just prior to MBS8(1V270) administration.

Occurrence of Cytokine Release Syndrome (CRS) was assessed on all dosing occasions. CRS was defined in accordance with Common Terminology Criteria for Adverse Events (CTCAE) version 5.0 as “a disorder characterized by fever, tachypnea, headache, tachycardia, hypotension, rash, and/or hypoxia caused by the release of cytokines”. In case CRS was observed it was graded according to CTCAE version 5.0 criteria for grading of CRS, as shown in Table 1.

Conclusion: There was no CRS observed at any dosing occasion in patient 4-7 which were pretreated with tocilizumab, whereas patient 1-3 that were not pretreated with tocilizumab all experienced CRS during the 4 administrations.

In total CRS was experienced at 0 of the 14 dosing occasions (0%) for the tocilizumab premedicated patients, but in 7 out of 12 occasions (58%) in the non-premedicated patients.

1. A combination drug comprising, separately or together, an anti-interleukin-6 (IL-6) agent and a CRS inducing immunotherapeutic agent for use in the treatment of a disease in a patient, wherein the anti-IL-6 agent is administered to the patient prior to, simultaneously with, or after the CRS inducing immunotherapeutic agent, wherein the patient does not present with symptoms of CRS; wherein the CRS inducing immunotherapeutic agent is a toll-like receptor (TLR) agonist.

2. The combination drug for use according to any one of the preceding claims, wherein the patient a) is at risk of developing cytokine release syndrome (CRS), such as drug induced CRS, and/or
b) suffers from a condition that increases the risk of developing CRS, such as a cancer, or an autoimmune disease.

3. The combination drug for use according to any one of the preceding claims, wherein the anti-interleukin-6 agent is an interleukin-6 receptor (IL-6R) blocking agent, an interleukin-6 (IL-6) blocking agent, or an interleukin-6 (IL-6) signalling blocking agent.

4. The combination drug for use according to any one of the preceding claims, wherein the anti-interleukin-6 agent is:
a) a monoclonal antibody against the interleukin-6 receptor (IL-6R), such as a humanized monoclonal antibody against IL-6R, or
b) a monoclonal antibody against interleukin-6, such as a humanized monoclonal antibody against IL-6.

5. The combination drug for use according to any one of the preceding claims, wherein the anti-IL-6 agent is selected from the group consisting of: tocilizumab (CAS: 375823-41-9), siltuximab (CAS: 541502-14-1), sarilumab (CAS: 1189541-98-7), olokizumab (CAS: 1007223-17-7), elsilimomab (CAS: 468715-71-1), clazakizumab (CAS: 1236278-28-6), and sirukumab (CAS: 1194585-53-9).

6. The combination drug for use according to any one of the preceding claims, wherein the CRS inducing immunotherapeutic agent is a toll-like receptor (TLR) agonist selected from the group consisting of: a TLR1 agonist, a TLR2 agonist, a TLR3 agonist, a TLR4 agonist, a TLR5 agonist, a TLR6 agonist, a TLR7 agonist, a TLR8 agonist, a TLR9 agonist, a TLR10 agonist, a TLR11 agonist, and a TLR12 agonist; or a dual agonist thereof, such as a dual TLR7/8 agonist.

7. The combination drug for use according to claim 6, wherein the TLR7 agonist is selected from the group consisting of: 852A; BNT411; DSP-0509; LHC165; NJH395; Resiquimod; RO7119929; TQ-A3334; BDC-1001; 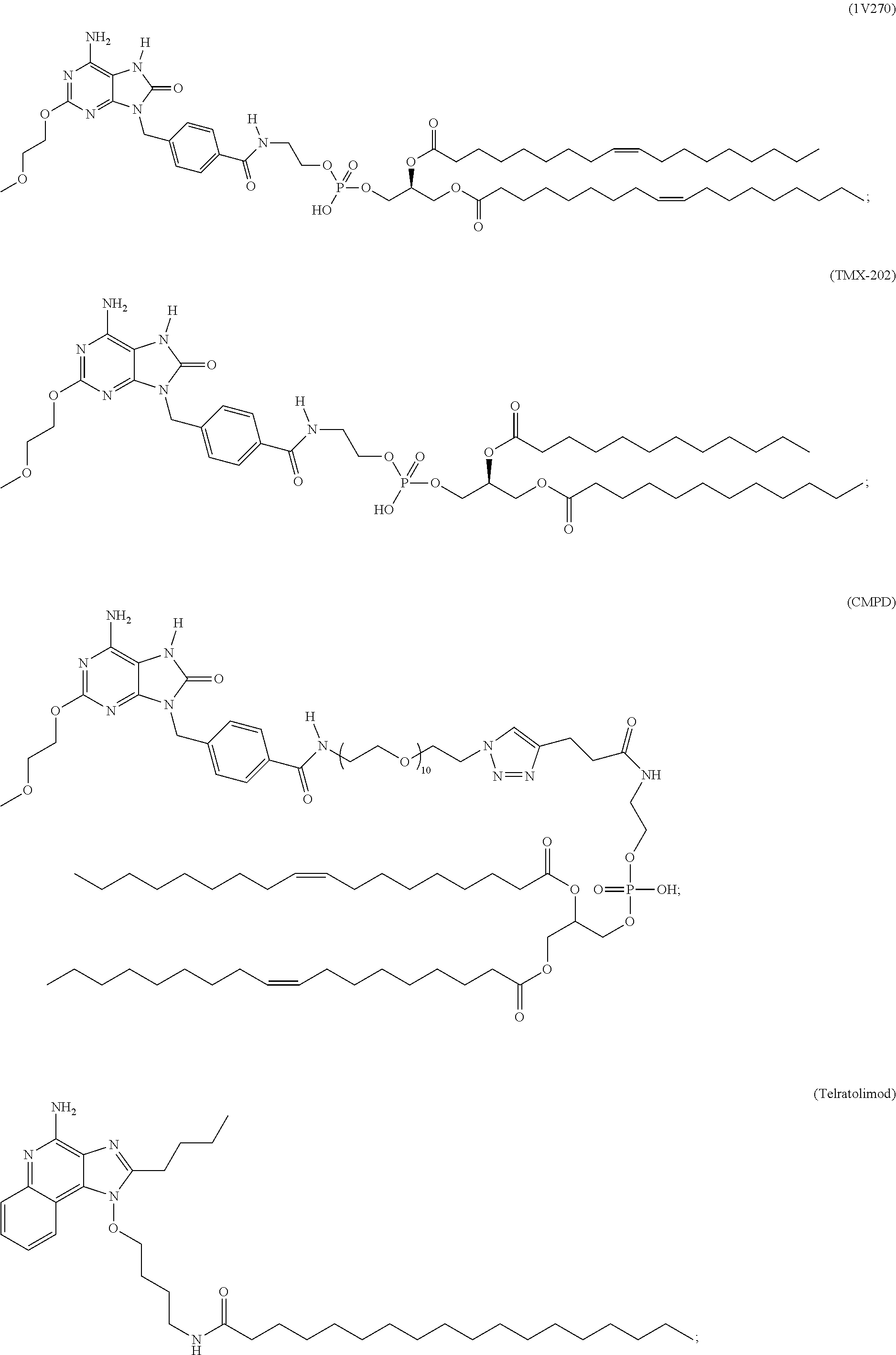 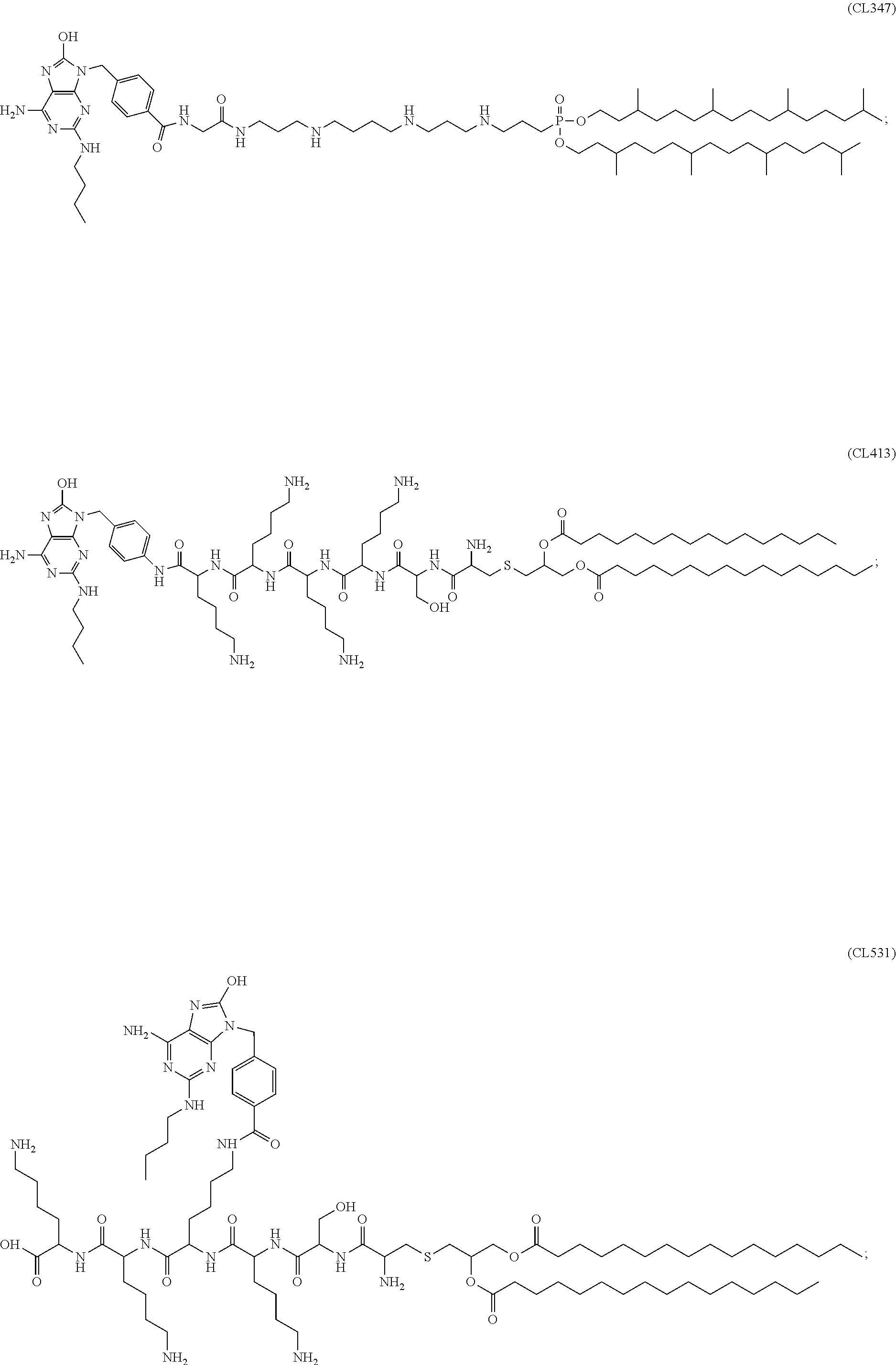 [SMILES: CCCCNc1nc(N)c2nc(O)n(Cc3ccc(C(=O)NCC(=O)NCCCNCCCCNCCCNCCCP(=O)(OCCC(C)CCCC(C)CCCC(C)CCCC(C)C)OCCC(C)CCCC(C)CCCC(C)CCCC(C)C)cc3)c2n1 ; CCCCCCCCCCCCCCCC(=O)OCC(CSCC(N)C(=O)NC(CO)C(=O)NC(CCCCN)C(=O)NC(CCCCN)C(=O)NC(CCCCN)C(=O)NC(CCCCN)C(=O)Nc1ccc(Cn2c(O)nc3c(N)nc(NCCCC)nc32)cc1)OC(=O)CCCCCCCCCCCCCCC ; CCCCCCCCCCCCCCCC(=O)OCC(CSCC(N)C(=O)NC(CO)C(=O)NC(CCCCN)C(=O)NC(CCCCNC(=O)c1ccc(Cn2c(O)nc3c(N)nc(NCCCC)nc32)cc1)C(=O)NC(CCCCN)C(=O)NC(CCCCN)C(=O)O)OC(=O)CCCCCCCCCCCCCCC]
a tautomer thereof, and a pharmaceutically acceptable salt thereof.

8. The combination drug for use according to claim 6, wherein the TLR8 agonist is selected from the group consisting of: CV8102; NKTR-262; and Motolimod.

9. The combination drug for use according to any one of the preceding claims, wherein the CRS inducing immunotherapeutic agent is formulated for parenteral or oral administration.

10. The combination drug for use according to any one of the preceding claims, wherein the CRS inducing immunotherapeutic agent is formulated as a micelle or a liposome, optionally wherein the micelle has a diameter of from 5 nm to 39 nm.

11. The combination drug for use according to claim 1, wherein the micelle is MBS8, comprising
i) 1,2-distearyl-sn-glycero-3-phosphoethanolamine-N-[methoxy(polyethylene glycol)-2000](DSPE-PEG2000); and
ii) the TLR7 agonist of formula (IA), 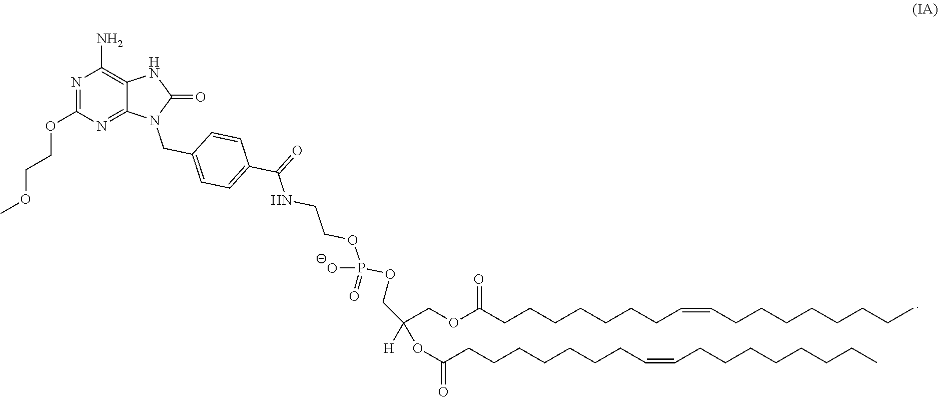 [SMILES: CCCCCCCC/C=C\CCCCCCCC(=O)OCC(COP(=O)([O-])OCCNC(=O)c1ccc(Cn2c(=O)[nH]c3c(N)nc(OCCOC)nc32)cc1)OC(=O)CCCCCCC/C=C\CCCCCCCC]
or a pharmaceutically acceptable salt or solvate thereof;
wherein the molar ratio of DSPE-PEG2000 to the TLR7 agonist 90:10.

12. The combination drug for use according to any one of the preceding claims, wherein the disease is a cancer, optionally wherein the cancer is selected from the group consisting of: a solid tumor and a liquid tumor.

13. The combination drug for use according to any one of the preceding claims, wherein the anti-IL-6 agent is administered within a predefined time interval from prior to administration of the CRS inducing immunotherapeutic agent to after administration of the CRS inducing immunotherapeutic agent.

14. The combination drug for use according to any one of the preceding claims, wherein the predefined time interval is from 240 minutes prior to administration of the CRS inducing immunotherapeutic agent to 240 minutes after administration of the CRS inducing immunotherapeutic agent.

15. The combination drug for use according to any one of the preceding claims, wherein the anti-interleukin-6 (IL-6) agent is tocilizumab (CAS: 375823-41-9), and the CRS inducing immunotherapeutic agent is MBS8 and wherein tocilizumab is administered to the patient from 240 minutes prior to administration of MBS8 to 180 minutes after administration of MBS8.
